(19) United States
(12) Patent Application Publication
(10) Pub. No.: US 2015/0038589 A1
(43) Pub. Date: Feb. 5, 2015

(54) Nutritional Product Comprising a Biguanide

(71) Applicant: Karel Paul Bouter, Vught (NL)

(72) Inventor: Karel Paul Bouter, Vught (NL)

(21) Appl. No.: 14/234,022

(22) PCT Filed: Jul. 22, 2011
(86) PCT No.: PCT/NL2011/050538

§ 371 Date: Mar. 19, 2014

(51) Int. Cl. A61K 31/155; A23K 1/18; A23C 9/123; A23K 1/16; A23L 1/30; A21D 2/24

(52) U.S. Cl. CPC A61K 31/155; A23L 1/30; A21D 2/24; A23C 9/123; A23K 1/1609; A23K 1/1806; A23V 2002/00
USPC 514/635

(57) ABSTRACT

The present invention relates to a nutritional product comprising a biguanide in a concentration of 20 to 1600 mg per 100 g, based on total weight of the nutritional product, the nutritional product further comprising at least one dietary component selected from the group of (i) digestible carbohydrates, (ii) amino acid sources, such as proteins, peptides, free amino acids including salts of free amino acids, (iii) lipids, and (iv) dietary fibre. The invention further relates to a nutritional product comprising a biguanide for use in a prophylactic or therapeutic treatment of an aging-associated disorder.

The invention relates to a nutritional product comprising a biguanide. The invention is further directed to a nutritional product comprising a biguanide for a specific use.

Changes in societies throughout the world, like reduction in exercise—e.g. as a result of increasing automation—and changes in dietary habits, contribute to a growing part of the population suffering from so called ‘disorders of civilisation’ (also known as ‘Western diseases’). Such disorders are often aging-associated. A subject is considered to have an aging associated disorder if one or more physiological parameters that are markers for a specific disorder that tends to either increase or decrease with age, are outside the normal range for a subject of a specific age, in that they are elevated if the parameter is one that generally increases with age (such as blood pressure). It decreases if the parameter is one parameter that generally decreases with age (such as activity of beta-cells).

Relevant parameters for various aging associated diseases and normal ranges for subjects of specific ages (taking into account factors like gender) are generally known and may for instance be found in The aging male, The official journal of the international society for the study of the aging male (Bruno Lunenfeld, ed), Parthenon Publishing (Volume 1, No 1, January 1998) or in Global burden of disease 2004 update WHO Global Burden of Diseases and Women, Ageing and Health. A framework for action WHO UPFPA 2007.

Global Burden of Disease analysis provides a comprehensive and comparable assessment of attributable mortality and loss of health due to diseases, injuries and risk factors for all regions of the world. The overall burden of disease is assessed using the disability-adjusted life year (DALY), a time-based measure that combines years of life lost due to premature mortality and years of life lost due to time lived in states of less than full health.

Leading causes of attributable mortality include high blood pressure, tobacco use, high blood glucose, physical inactivity, overweight, obesity, high cholesterol, unsafe sex, alcohol use, childhood underweight, indoor smoke from solid fuels. Leading causes of attributable DALYs include childhood underweight, unsafe sex, alcohol use, unsafe water, sanitation, hygiene, high blood pressure, tobacco use, suboptimal breast feeding, high blood glucose, indoor smoke from solid fuels, overweight and obesity. Further important risk factors for attributable DALYs include insufficient consumption of fruit and/or vegetables, insufficient exercise, high cholesterol.

Ageing is a continuous process beginning at the conception. There are several factors which will influence the ageing of the individual during his live. These factors are genes, nutrition, surrounding and lifestyle. Persons who eat too much and have insufficient exercise will gain overweight; rising of their blood pressure, overweight and an increased blood pressure will induce the aging process.

Examples of disorders of civilisation which often are aging associated are obesity, cardiovascular diseases, arthrosis, (weight-related) hypertension, hypercholesterolemia, insulin resistance, metabolic syndrome, cancer and diabetes type II.

An article in the Lancet vol 378, pp 2215-2222, 2011 showed that in 2008, a global age standardised mean fasting plasma glucose was 5.5 mmol/l for men and 5.42 mmol/l for woman, having rising by 0.07 mmol/l and 0.09 mmol/l per decade respectively. Age standardise adults diabetes prevalence was 9.8% in men and 9.2% in woman in 2008 up from 8.3% and 7.5% in 1980. Glycaemia and diabetes are rising globally, driven both by population grow and ageing by increasing age specific prevalence.

The Decode study published in the Diabetes Metab 2000, September, 26(4) 282-286 showed a direct relation between rising blood sugar and mortality especially cardiovascular mortality. The Emerging Risk Factors Collaboration published in June 2011 pp 829-840 in the New England Journal of Medicine that patients with fasting glucose levels exceeding 5.6 mmol/l were associated with death. A 50 year old person with diabetes will die, on average, 6 years earlier than a counterpart without diabetes, with about 40% of the difference in survival attributable to an excess of nonvascular deaths like cancer of the liver, pancreas, ovary colorectal, lung, bladder and breast.

In the same publication the found that people without diabetes but with rising fasting glucose with conventional risk factors like hypertension have in improved risk on cardiovascular diseases.

Effectiveness of a specific therapy depends on many factors, including gender, various genetic factors, age, ethnic background, and life-style.

Thus, there is a strong need for various alternative methods of prevention and treatment of subjects suffering from such a disorder, in order to cure the subject or at least reduce the risk of developing complications or reduce the risk of developing a further disorder of civilisation (which may be more severe), such as hypertension. or increasing fasting glucose.

Furthermore, programs aimed at reducing the risk of developing such disorders by promoting a more healthy life-style have been proposed. However, these are only partially successful and have—so far—not been able to reduce the number of new patients with aging associated lifestyle disorders like diabetes, hypertension, dyslipedaemia and atherosclerosis.

It is an object of the invention to provide a product for use in a prophylactic or therapeutic treatment of an aging associated disorder, in particular insulin resistance or a disorder starting with insulin resistance and progressing to one or more disorders selected from hypertension, dyslipedaemia, prediabetes and atherosclerosis, that may serve as an alternative to known products for such use or as an alternative to known methods of addressing aging associated disorder.

It is a particular challenge to provide a method for therapeutically treating a subject suffering from a aging associated disorder, or to provide a method for prophylactically treating a subject to reduce the risk of developing an aging associated disorder, in cases wherein the subject is not aware of having any (significant) symptoms of the disorder. Generally, such subjects are not very motivated to comply with treatment instructions and often skip parts of the treatment or discontinue their treatment prematurely. Such lack of subject-compliance is even increased if the treatment causes discomfort, as may be the case if the treatment involves administration of a drug (due to undesirable side-effects, such as gastro-intestinal problems) or performing specific activities.

Thus, it is in particular an object of the invention to provide a product suitable for use in the prophylactic or therapeutic treatment of an aging associated disorder, which shows a satisfactory subject-compliance.

One or more specific advantages of the invention will be apparent from the description following below.

It is the inventor's finding that this object is met by a nutritional product comprising a specific active compound.

Accordingly, the present invention relates to a nutritional product comprising a biguanide (as active ingredient), and optionally at least one component selected from vitamin D (as active ingredient) and a pectin (as active ingredient) for use in the treatment of an aging associated disorder.

In particular, the invention relates to a nutritional product for use in a disorder selected from the group of insulin resistance, diabetes type II, metabolic syndrome, obesity, hypertension, hypercholesterolaemia, dyslipaedea, atherosclerosis, steotosis (fatty liver), cancer (in particular lung carcinoma, liver carcinoma or colonic carcinoma), and polycystic ovary syndrome.

In particular, the invention relates to a nutritional product comprising a biguanide (as an active ingredient) for use in a prophylactic treatment of a subject to reduce the risk of developing hyperinsulinaemia or insulin resistance or for use in the therapeutic treatment of insulin resistance or hyperinsulinaemia.

In particular, the nutritional product comprising a biguanide (as an active ingredient) may be for use in the treatment of a non-diabetic subject to reduce the risk of developing a disorder selected from the group of diabetes type II, hypertension, dyslipaedea, atherosclerosis and steatosis (fatty liver).

In particular, the present invention relates to a nutritional product comprising a biguanide (as an active ingredient) for use in the treatment of obesity.

In particular a nutritional product (may be used in accordance with the invention to reduce visceral fat content of a subject.

Further, the invention relates in particular to a nutritional product comprising a biguanide, usually in a concentration of 20-1600 mg per 100 g, based on total weight of the nutritional product, in particular in a concentration of 80-1200 mg per 100 g, based on total weight of the nutritional product, more in particular in a concentration of 250-1000 mg/100 g, based on total weight of the nutritional product. A preferred concentration may be selected depending on the type of product and the desired daily dosage. For example, in a product that is typically consumed in a relatively low amount per dosage the concentration can be relatively high, in particular above 1000 mg/100 g, based on total weight of the nutritional product. An example of such a product is a cookie. A product that is typically consumed in a relatively high amount, in particular a dairy product such as yoghurt or the like, or a dairy-substitute product, the concentration may be relatively low, in particular 500 mg/100 g or less, based on total weight of the nutritional product. more in particular 80-360 mg/100 gram.

Further, the invention relates to a kit of parts, comprising a first unit dose packaging containing a nutritional product and a second unit dose packaging containing a biguanide, preferably metformin, and optionally instructions for adding the biguanide to the nutritional product. The nutritional product in a kit of parts according to the invention is usually a nutritional product as defined elsewhere in the present disclosure, with the proviso that it will generally be free of biguanide, when still packaged in its unit dose packaging. The kit of parts may in particular be for any of the above mentioned uses, more in particular for use in the prophylactic or therapeutic treatment of insulin resistance or hyperinsulinaemia

Further, the invention relates to a method of treating a subject, the method comprising oral administration of a nutritional product (for use) according to the invention.

The term “or” as used herein means “and/or” unless specified otherwise.

The term “a” or “an” as used herein means “at least one” unless specified other wise.

When referring to a noun (e.g. a compound, an additive, etc.) in the singular, the plural is meant to be included. Thus, when referring to a specific moiety, e.g. “compound”, this means “at least one” of that moiety, e.g. “at least one compound”, unless specified otherwise.

The term ‘treatment’ as used herein includes prophylactic treatments and therapeutic treatments, unless specified otherwise.

Studies by the inventors suggest that a biguanide (metformin) can be included in a nutritional product, also when subjected to a heat treatment (at least up to 200° C., e.g. baking or a pasteurisation/sterilisation), and is storage stable for at least several weeks, in particular for at least 5 weeks (for a product typically having a storage stability for such a period). It has also surprisingly been found that an acidic composition according to the invention has a good storage stability.

For a good storage stability the product may alternatively be provided is a kit of parts. This may be advantageous in view of prolonged storage stability. Further this may be desired, at least in some jurisdictions in view of food and drugs regulations.

Usually, the nutritional product further comprises at least one dietary component selected from the group of (i) digestible carbohydrates, (ii) amino acid sources, such as proteins, peptides, free amino acids including salts of free amino acids, (iii) lipids and (iv) dietary fibre. In particular, at a least a digestible carbohydrate and an amino acid source may be present. A nutritional product according to the invention is particularly suitable for use in any one or more of the above mentioned treatments.

In addition to being effective for use in one or more of the treatments identified in the present disclosure, such as prophylactic or therapeutic treatment of insulin resistance, a nutritional product or kit of parts (for use) according to the invention may have one or more additional advantages.

In particular, it is considered that the nutritional product or kit of parts according to the invention shows satisfactory subject-compliance. Subject-compliance is the tendency of the subject to continue treatment with (a composition comprising) a pharmaceutical substance. Lack of subject-compliance is often a problem when subjects are treated with a biguanide, in particular metformin. In accordance with the invention it is considered that satisfactory subject-compliance can be reached also if a subject is to be treated for a long period, e.g. for at least 6 weeks, or chronically.

Further, a nutritional product or kit of parts (for use) according to the invention may help to stabilise the body weight of a subject or to reduce body weight gained over a prolonged period, such as a over a period of 1 year or more.

It is a particular advantage of the invention that it allows treatment, in particular a prophylactic or therapeutic treatment of insulin resistance, without needing to change the diet of the subject to be treated. The nutritional product may simply replace a comparable product the subject is already used to eating. Further, the product is effective without needing to increase daily physical exercise or having any other change in life-style of the subject. Accordingly, in an advantageous embodiment, the nutritional composition is used in a treatment that does not involve a change in diet (other than taking the nutritional composition of the invention) and/or that does not involve increasing daily physical exercise.

It is observed that biguanides are known pharmaceutically active compounds that can be used for instance for the therapeutic treatment of diabetes.

As used herein, the term ‘biguanides’ generally encompasses compounds represented by the formula:

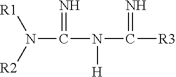

and salts thereof that are suitable for use in a food or pharmaceutical application. Suitable salts include the chloride salt (biguanide HCl). Herein R1 and R2 independently represent a hydrogen atom, an alkyl group, a cycloalkyl group, a heterocycle, and alkenyl group, an aryl group, an aralkyl group, an aryloxyalkyl group or a heteroaryl group, or R1 and R2 taken together represent an alkylene, which may comprise one or more hetero atoms.

R3 represents a primary, secondary or tertiary amine. R3 may in particular be a primary amine. In case R3 represents a secondary or tertiary amine, the substituents for the hydrogens may in particular be a C1-C7 alkyl.

Such compounds have, e.g. been described in U.S. Pat. No. 7,199,159.

Said alkyl group may in particular be a C1-C7 alkyl, such as methyl, ethyl or propyl.

Said cycloalkyl group may in particular be a C3-C7 cycloalkyl, such as cyclohexyl.

Said heterocycle may in particular contain a ring structure of 3 to 7 carbons and at least one heteroatom selected from O, S and N. The alkenyl group may in particular be a C2-C7 alkenyl, such as vinyl or allyl.

Said aryl group may in particular be an aromatic hydrocarbon ring, such as a phenyl group, which is unsubstituted or substituted with a substituent selected from C1-C7 alkyl groups, C2-C7 alkenyl groups, Cl, I, Br and F. The aryl is either directly attached to the remainder of the compound or by a linker, such as an —NH—, an alkyl (in which case the group may be referred to as aralkyl) or an oxyalkyl (in which case the group may be referred to as aryloxy).

Said aralkyl group may in particular be an aryl, as mentioned above, linked to the remainder of the compound via a —(CH₂)ₙ— with n being an integer of 1 to 7.

Said aryloxyalkyl group may in particular be an aryl, as mentioned above, linked to the remainder of the compound via a —O—(CH₂)ₙ— with n being an integer of 1 to 7

Said heteroaryl group may in particular be an unsubstituted or substituted aromatic ring, such as a six-membered aromatic ring, comprising at least one heteroatom selected from S, O and N. The substituents may in particular be as specified for the aryl.

If R1 and R2 taken are together, they may in particular form a C2-C7 alkylene which may comprise one or more heteroatoms selected from O and S.

In a preferred embodiment, the biguanide is selected from biguanides wherein R1 and R2 independently represent a methyl, an ethyl or a propyl.

In a specifically preferred embodiment, the biguanide in a nutritional product of the invention is metformin (also known as N,N-dimethylimidodicarbonimidic diamide or 1,1-dimethylbiguanide).

In principle, a product according to the invention may be used by any animal. A product according to the invention is in particular suitable for treatment of humans or other mammals. In a preferred embodiment, the subject is selected from humans, dogs, horses and cats. Other mammals which in particular may be treated include rabbits, cows and pigs.

Dogs may in particular benefit from treatment in accordance with the invention wherein the risk of developing insulin resistance or obesity is reduced.

Horses may in particular benefit from treatment in accordance with the invention wherein insulin resistance is prophylactically or therapeutically treated. In particular treatment of a horse, in accordance with the invention may be used to reduce the risk of the horse becoming cripple, becoming obese or developing laminitis or to therapeutically treat crippleness, obesity or laminitis. Crippleness and laminitis may be symptoms of insulin resistance, in horses.

The horse may be of any race, in particular of one of the following races: Shetlandpony's, Welsh pony's and Quarter Horses.

A horse to be treated with a composition according to the invention may in particular be an insulin restant horse. The horse may in particular meet at least one of the following criteria: being overweight or obese, having an increased fasting insulin level (typically more than 20 mU/l, and having an increased fasting glucose level (typically more than 5.5 mmol/l). A preferred daily dosage for a horse to be treated with a composition according to the invention is in the range of 1000 to 3000 mg. The nutritional composition can be regular daily feed for a horse or a treat for a horse to which the biguanide, in particular metformin, has been added. Preferably, the feed also contains (added) pectin and/or (added) vitamin D. An effective amount thereof may be based on the information disclosed herein, common general knowledge and optionally some routine testing.

In a specific embodiment, the nutritional product in the unit-dose package contains 1000-3000 mg biguanide and a horse feed (mixed or provided as a kit of parts).

A product according to the invention is in particular suitable for male adults, female adults, male adolescents or female adolescents. For humans, the term ‘adults’ is used herein for humans having reached an age of 18 years or more. For humans, the term ‘adolescents’ is used herein for non-adults of at least 11 years.

A composition according to the invention may be administered to a human of any ethnic background, e.g. Caucasian or Asian.

A human subject preferably meets one or more of the following criteria:

the subject is overweight, typically having a body mass index (BMI) of more than 25, preferably of 26.0 or more, in particular the subject is obese (a BMI of 30 or more, more in particular of 35 or more). BMI equals body weight (in kg) divided by length squared (in m²).

for adults: the subject with an increased waist size (i.e. of over 92 cm for males or of over 82 cm for females).

the subject—with one or more relatives (first, second or third degree) with diabetes type II, in particular at least one relative of the first degree with diabetes type II.

the subject suffering from diabetes II, and treated with insulin (this criterion is of course not relevant for a treatment directed at preventing diabetes)

subjects with insulin resistance, unless the product is for use in the prevention of insulin resistance,

the subject having hypertension (unless the product is for use in the prevention thereof)

the subject having dyslipedaemia (unless the product is for use in the prevention thereof)

the subject having polycystous ovary syndrome (unless the product is for use in the prevention thereof)

the subject having a carcinoma (unless the product is for use in the prevention thereof)

the subject having beta-cell failure or a progressive beta-cell loss rate (>1% loss per year), (unless the product is for use in the prevention thereof)

the subject not having impaired glucose intolerance (the subject having a fasting glucose level in blood of 6.0 mmol/l or less), unless the product is for therapeutically treating impaired glucose intolerance.

the subject being non-diabetic.

For determining whether a subject has insulin resistance, the homeostasis model assessment (HOMA), e.g. the HOMA calculator, available from the University of Oxford can be used (http://www.dtu.ox.ac.uk/homacalculator/index.php).

As used herein, a subject who is insulin resistant is in particular defined as an overweight subject, the subject further having an increased fasting glucose level (at least 6-7 mmol/l) and/or an increased insulin to glucose ratio.

In humans, the insulin to glucose ratio is in particular considered to be increased if the insulin to glucose ratio (HOMA-IR) is above 2 IU/mmol.

In a specific embodiment, an insulin resistant subject meets one or more of the following criterions: increased waist size (male >92 cm; female >82 cm), increased fasting blood triglycerides (>1.69 mmol/l), reduced fasting HDL (<1.0 mmol/l) increased fasting LDL cholesterol (>2.5 mmol/l, increased blood pressure (>135/85 MmHg).

Specific effects that are indicators for an effective treatment in accordance with the invention may in particular include one or more of the following:

a reduction in waist girth (without a change in daily caloric intake)

a reduction in body weight

a reduction in visceral fat

a reduction in systolic blood pressure

a reduction in diastolic blood pressure

a reduction in fasting blood-glucose level

a reduction of insulin levels

a reduction in average blood-glucose level (as determined by measuring the glycated haemoglobin (HbA1c) level)

a decrease in blood-triglyceride level

an increase in Hdl-cholesterol

(all changes compared to the situation just before the start of the treatment)

A product used for the treatment of insulin resistance is in particular considered to be effective if over a period of treatment of about six weeks one or more of the following effects have occurred: A reduction in HbA1c level in blood of at least 2.0 mmol/ml a reduction in fasting blood-glucose level by at least 0.1 mmol/l, preferably by at least 0.2 mmol/l; a reduction in weight of at least 1.5 kg, waist size reduction of at least 1 cm (visceral fat reduction), and reduction of systolic pressure of at least 3 mmHg

Regarding uses of a product according to the invention for the prevention of a medical indication, it is observed that the effectiveness can generally be determined by long-term studies of subjects not suffering from said medication. The subjects are divided into two groups. The first group receives the product according to the invention and the second group receives a placebo, in order to investigate whether in the first group the incidence of the medical indication is significantly less. Suitable procedures are generally known in the art. It is also possible to monitor one or more parameters relevant to developing the medical indication and determine whether the product has a positive effect on these parameters. For instance for insulin resistance, a reduction in the insulin to glucose ratio by administration of the product is an indication that the product is effective for preventing insulin resistance. This also is an indication that the product is effective in prevention of disorders like atherosclerosis, diabetes, hypertension and dylipidaemia.

A composition for use in accordance with the invention is usually administered at least three times per week, preferably—on average—at least once a day, e.g. up to three times a day.

The (average) daily dosage in a nutritional product (for use) according the invention or in a dose unit packaging of a kit of parts according to the invention is usually chosen lower than generally dosed when using the biguanide for a therapeutic treatment of a diabetic patient. The (average) daily dosage is usually below 850 mg. Preferably, the (average) daily dose is 500 mg or less, in particular 475 mg or less, more in particular 450 mg or less, 400 mg or less or 375 mg or less. The (average) daily dosage usually is at least 50 mg, in particular at least 150 mg, more in particular at least 200 mg, at least 250 mg, at least 275 mg, at least 300 mg or at least 350 mg. These dosages in particular apply to the treatment of an (adult) human. If treating another mammal or a non-adult human, a different dosage may be applied.

As an alternative, the daily dosage may be based on body weight of the subject. Preferably, the (average) daily dose, based on body weight, is 6.25 mg/kg or less, in particular 5.6 mg/kg or less, 5.0 mg/kg or less or 4.4 mg/kg or less. The (average) daily dosage, based on body weight, usually is at least 0.6 mg/kg, preferably at least 1.2 mg/kg, in particular at least 1.9 mg/kg, more in particular at least 2.5 mg/kg or at least 3.1 mg/kg.

In a specific embodiment that dosage is a dosage that is effective in providing a metformin concentration in whole blood in the range of 0.5-2.0 mg/l, preferably 1.0-2.0 mg/l, approximately 1 to 4 hours after consumption, in particular within about 2 to about 4 hours after consumption.

A treatment according to the invention may be continued for a prolonged period, usually for at least 6 weeks, in particular for at least 3 months and may be continued for up to a year or for more than a year, e.g. for the rest of the subject's life. In case the subject has a high BMI (>25) or a high waist size (>92 cm for human males or >82 cm for human females), the treatment may be discontinued after the BMI and/or waist girth has been reduced below said values. Alternatively, the treatment may prophylactically be continued, e.g. to avoid the subject becoming obese (again).

The nutritional product may be any product suitable as nutrition for a specific subject. The product can be a food product forming part of a normal diet, e.g. bread, (substitute) yoghurt, (substitute) butter. Also it can be provided as a complete nutrition, especially in case a mammal other than a human is treated, or as a treat (candy, snacks).

Besides the biguanide, such as metformin, a nutritional product (for use) according to the invention or in a kit of parts according to the invention in general comprises one or more nutritional ingredients. In general, the nutritional ingredients of a nutritional product according to the invention may be selected from known nutritional ingredients for a specific product.

Preferably one or more nutritional ingredients selected from the group of (i) digestible carbohydrates, (ii) amino acid sources, such as proteins, peptides, free amino acids including salts of free amino acids, (iii) lipids and (iv) dietary fibre are present. Further, one or more components selected from the group of minerals, trace elements, vitamins and further micro-nutrients, e.g. antioxidants, may be present. Further, the product may comprise any food additive, in particular one or more food additives selected from the group of colourants, flavours, aroma's, acids, preservatives, stabilisers, thickening agents and the like.

Advantageously the nutritional product, in particular a dairy product or dairy substitute product, such as yoghurt or a yoghurt-substitute, or a nutritional product for a horse, comprises vitamin D.

In a preferred embodiment, the vitamin D is present in the nutritional product in a concentration to provide a daily dosage (administered via the nutritional composition or kit of parts of the invention) in the range of 150-750 IU (international units, 5-15 micrograms equivalent), in particular in the range of 250-600 IU. In particular in a dairy or dairy substitute product, the vitamin D concentration preferably is in the range of 120-600 IU/100 gram, more preferably in the range of 200-480 IU/100 gram. Vitamin D may in particular be used to increase the insulin sensitivity in combination with the biguanide. Advantageously, pectin is present in a dairy product, in particular yoghurt or a yoghurt substitute according to the invention. Pectin is particularly suitable for stabilising a biguanide dispersed or dissolved in a fluid product. Further, pectin may have a beneficial effect on the intended use of the composition. In particular, pectin may contribute to decreasing cholesterol level. Also, a pectin may contribute to increase insulin sensitivity.

Preferably, the nutritional product comprises a pectin selected from the group of pectins comprising one or more galacturonic acid units, such as pectins from apple.

Preferably, the pectin is present in the nutritional product in a concentration to provide a daily dosage (administered via the nutritional composition or kit of parts of the invention) of at least 0.1 gram, in particular of at least 0.2 gram, more in particular of at least 0.4 gram. Usually, the dosage (administered via the nutritional composition or kit of parts of the invention) is 20 gram per day or less, in particular 10 gram per day or less, more in particular 2 gram or less.

The pectin concentration in a product according to the invention is preferably in the range of 0.1-4 wt. %, in particular 0.3-2 wt. %, more in particular 0.4-1 wt. %.

In an advantageous embodiment, the product comprises juice of a citrus fruit, such as lemon juice. This has been found advantageous with respect to neutralising the taste of the biguanide, which may be experienced as unpleasantly bitter. Another possibility to achieve this is to provide the product with one or more food-grade organic acids, which may be selected from the group of citric acid, malic acid and tartaric acid. The organic acid or fruit juice are usually present in an effective amount to neutralise the taste of the biguanide. An effective amount may be determined empirically, depending on the amount of biguanide and the composition of the product. In particular citric acid is, preferred for a nutritional composition according to the invention to provide a product with a pleasant taste. The citric acid may be added as a fruit juice, e.g. lemon juice in an amount of 0.01-0.5 wt. %, in particular 0.02-0.2 wt. % lemon juice relative to the weight of the total product.

Further, a nutritional product according to the invention may comprise one or more probiotics, e.g. selected from the group of lactic acid bacteria and bifidobacteria.

In an advantageous embodiment the nutritional product according to the invention, is selected from the group of fermented milk products, unfermented milk products, fermented milk-substitute products, and unfermented milk-substitute products.

As used herein, the term ‘milk-substitute product’ or ‘dairy-substitute product’ is used for products that are made of vegetable materials and can be used to emulate or replace a dairy product, such as soy-milk, rice milk, almond milk etc.

Preferred examples of fermented products include yoghurts and drinks comprising fermented milk or yoghurt (in the art also referred to as yoghurt drinks), e.g. kefir, lassi or ayran. A fermented milk product, such as yoghurt, is in particular preferred for use in a treatment according to the invention. Herein, when referred to ‘yoghurt’ this term is meant to include yoghurt-substitute and yoghurt drinks, unless specified otherwise. Besides providing an effective matrix for the active compound, it is considered that the risk of gastro-intestinal side-effects, that can be caused by biguanides, such as metformin, is relatively low. Further, a fermented dairy product (or substitute thereof) according to the invention is considered to be advantageous in that one or more other symptoms that have been reported in the prior art to occur when administering a biguanide, such as excess hunger, giddiness, excess sweating, stomach complaints, diarrhoea, can be avoided or occur to a low extent. In particular, absence or at least low incidence of stomach complaints and diarrhoea is surprising, because—typically—roughly about 30% of subjects treated with a conventional pharmaceutical composition comprising biguanide (metformin) suffer from such a side-effect.

An advantageous side-effect that has been found in a clinical test is reduced appetite, whereby a composition according to the invention may help to manage the daily caloric intake at an acceptable level, whereby the dairy product may be particularly advantageous for therapeutic treatment or prophylaxis of an aging associated disease, such as insulin resistance, diabetes type II or obesity, in accordance with the invention.

Further, the inventors have come to the conclusion that biguanide, in particular metformin, administered as part of a yoghurt, has improved pharmacological effectivity, compared to metformin adminstered as, e.g. a tablet. In particular, it is contemplated that it provides an improved effect on glucose level in blood, and/or reduces the incidence of negative side-effects on the B12 levels in blood after administration of biguanide (in conventional dosage form such as a tablet, metformin causes a reduction in B12 levels, in about 30% of the subjects). It is further surprising that good gastro-intestinal resorption of biguanide is achieved when administered in a fermented product, which is acidic, since acidity often has an adverse effect on gastro-intestinal resorption of pharmaceutical products.

Also it has been found that subject-compliance is good. Further, in an embodiment, it has been found that a disadvantageous taste experience, in particular a metallic taste that may be caused by biguanide, is avoided in a fermented milk product. In a clinical test the product taste has been marked as good.

A preferred example of a non-fermented milk product respectively substitute thereof is butter respectively margarine, in particular butter respectively margarine for use as a spread on bread.

In a further advantageous embodiment, the nutritional product is a cereal product, in particular a baked cereal product, such as a product selected from cookie, cakes and granola bars.

In a further embodiment, the nutritional product is a fruit drink

In a specific embodiment, the nutritional product has a lipid content of less 0-6 wt. %, in particular 3 wt. % or less, more in particular 1 wt. % or less. Obviously, such a product will not be butter or margarine.

The nutritional product may in principle comprise any edible lipid. Usually, unless the nutritional product is lipid-free, it comprises a mixture of lipids. A lipid in a product of the invention may be of vegetable or animal origin. Examples of lipid-mixtures of vegetable origin are vegetable oils (from which fractions may be used). Suitable oils include corn oil, cottonseed oil, canola oil, olive oil, peanut oil, safflower oil, soybean oil, sunflower oil, oils from nuts and oils from fruit seeds. Suitable lipids from animal origin include milk-fat and fractions thereof.

The nutritional product may in particular comprise one or more in triglycerides. A triglyceride in a product according to the invention may be saturated, mono-unsaturated or poly-unsaturated. Preferred polyunsaturated fatty acids include omega-3 and omega-6 polyunsaturated fatty acids. Examples of omega-3 polyunsaturated fatty acids include include alpha-linolenic acid (ALA), eicosopentaenoic acid (EPA) docosapentaenoic acid (DPA), and docosahexanoic acid (DHA).

In a specific embodiment, 50-100 wt. %, in particular 60-98 wt. %, more in particular 70-95 wt. % of the lipids in a product according to the invention is formed by saturated and mono-unsaturated triglycerides.

If present, the polyunsaturated fatty acid content may in particular be in the range of 1-50 wt. %, more in particular in the range of 2-40 wt. % or in the rang of 5-30 wt. %.

In a specific embodiment, the digestible carbohydrate content is relatively low, in particular less than 50 wt. % based on total compounds contributing to caloric value of the product (i.e. digestible carbohydrates, proteins, peptides and amino acids, lipids), more in particular less than 40 wt. % or less, e.g. 1-30 wt. %, based on total compounds contributing to caloric value of the product.

Advantageously, the nutritional product is packaged in a unit-dose packaging, preferably a sealed packaging. Preferably, the nutritional product in the unit-dose package contains 50-500 mg, in particular 150-450 mg, more in a particular 250-450 mg biguanide. In principle, a nutritional product according to the invention may be prepared based on known methodology. For instance, the biguanide may be blended with an existing food product, made in a conventional way, for instance a liquid or semi-liquid nutritional product, or be blended with raw materials or intermediate materials which blend is subsequently used for the preparation of a known food product. E.g. the biguanide may be blended with dough (or the materials for forming a dough), after which the dough is used for preparing a baked food product.

A yoghurt or substitute yoghurt according to the invention is advantageously made in a specific way. Accordingly, the invention further relates to method for preparing a yoghurt or yoghurt-substitute according to the invention, wherein yoghurt or substitute-yoghurt is mixed with a biguanide stabiliser, thereafter the biguanide is added to the resulting mixture, and thereafter aseptically packaging the yoghurt comprising biguanide and biguanide stabiliser.

The stabiliser preferably is a pectin, which is usually added as an aqueous solution, in particular when a (substitute) yoghurt drink is prepared.

The (substitute) yoghurt is preferably subjected to a pasteurisation or sterilisation treatment. This is usually done after adding the stabiliser but before adding the biguanide.

The (substitute) yoghurt is preferably subjected to a homogenisation treatment. This is usually done after adding the stabiliser but before adding the biguanide.

One or more other ingredients may be added, in particular vitamin D and/or one or more flavours. This is usually done before homogenisation and/or sterilisation/pasteurisation, if any of such treatments are carried out.

After all ingredients have been added to the (substitute) yoghurt, the yoghurt or substitute-yoghurt comprising biguanide is aseptically packaged and sealed.

This method is in particular suitable to provide a (substitute) yoghurt having a good microbiological quality, with a good storage stability.

One or more other ingredients, in particular one or more ingredients selected from the group of vitamin D and flavours may be added. These are usually added before sterilisation or pasteurisation. The invention will now be illustrated by the following examples.

### EXAMPLE 1

### Cookie Comprising Metformin

A cookie dough was prepared comprising 1000 mg fine-ground metformin per 100 g conventional cookie dough, by mixing fine-ground metformin into the dough. 40 droplets of lemon juice per 100 g dough were added to the dough comprising the metformin, in order to neutralise the bitter taste of the metformin. Thereafter, the dough was divided in pieces of 25 g, from which pieces cookies were baked in a conventional way.

Human volunteers each consumed one cookie (providing 250 mg metformin) on an empty stomach. Blood samples were taken before consumption, 1 hour after consumption, 2 hours after consumption and 4 hours after consumption. Glucose levels, lipid levels and metformin levels in the samples were determined. In the glucose levels and in the lipid levels no significant changes were observed. The results for metformin are shown in the following table:

TABLE 1
metformin concentration (mg/l) in blood samples

| Time | subject 1 | subject 2 |
|---|---|---|
| before consumption | <0.5 | <0.5 |
| 1 hr after consumption | <0.5 | <0.5 |
| 2 hrs after consumption | 0.7 | <0.5 |
| 4 hrs after consumption | 0.8 | 0.6 |

These results show that metformin included in a food product (a product of which the production comprises a severe heating step), is absorbed by subjects consuming the food product to the extent that significant metformin levels are found in blood of said subjects.

During the experiment, no negative side-effects were observed.

### EXAMPLE 2

### Yoghurt Comprising Metformin

A yoghurt drink is made by mixing fine-ground metformin into fat-free yoghurt in an amount of 450 mg metformin per 100 ml yoghurt. 10 droplets of lemon juice per 100 ml yoghurt are added to neutralise the metformin taste.

### EXAMPLE 3

### Clinical Test

Preparation of Yoghurt Dosage Unit

A yoghurt-drink was prepared having the following composition:

73 wt. % skimmed yoghurt (5.5. % protein) (supplied by Campina, the Netherlands))

0.4 wt. % Grindsted® pectin AMD783 (Danisco, Czech Republic)

0.25 wt. % sweetener (Natrena)

0.03 wt. % flavour (lemon) (Givaudan, Switzerland)

26.3 wt. % water

This drink was prepared as follows.

To a weighed amount of the yoghurt the sweetener was added (Natrena).

Then the pectin and water were added as a pectin solution in water.

Then flavour was added.

Thereafter, the yoghurt was pasteurised in-line (Combitherm), using the following conditions: pre-heating to 65° C., homogenising at 65° C. (two-step: 180/18 bar), heating to about 90° C. for about 5 sec, cooling to 20° C. and aseptically packaging in 125 g dosage packaging (PET bottles).

Preparation of Metformin Dosage Unit

Metformin powder was made by crushing metformin tablets (BasicParma, Geleen, the Netherlands) each providing 850 mg metformin). Metformin powder was packaged in sachets, providing 450 mg metformin (for trial group I), or 250 mg metformin (for trial group II).

Sachets comprising for a control group were also provided. These sachets comprise microcrystalline cellulose.

Further, instructions were made to mix the contents of the sachets into the yoghurt.

Set-Up of the Trial

The trial is a double-blind trial. About 60 human subjects are divided into three groups of about 20 persons. Subjects in trial group I are treated with the yoghurt (125 g/day) to which a daily dosage of 250 mg metformin has been added, subjects in trial group II are treated with the yoghurt to which a daily dosage of 450 mg metformin has been added. The control group is treated with the yoghurt to which a placebo has been added.

The yoghurt is consumed on an empty stomach for a period of 6 weeks in a daily amount of 125 g per volunteer.

The human subjects have a body mass index of more than 26, are aged between 18 and 70, and have a gird of more than 92 cm (men) or a gird of more than 82 cm (women).

The human subjects are not diagnosed with diabetes mellitus; have a blood pressure that does not exceed 150/90 mmHg; have not been diagnosed with hypertension; have a cholesterol level that does not exceed 6.5 mmol/l; have not been diagnosed with familiar hypercholesterolaemia; have not been diagnosed with liver or kidney failure. The alcohol consumption of the subjects is less 20 E or less.

Throughout the study, and in the period of two weeks preceding the consumption of the first daily dosage, the subjects should maintain their regular diet. They shall not use any appetite depressants nor any fat absorption blockers or the like.

On the day preceding the first dosage, the subjects are given 21 sachets and 21 containers containing the yoghurt. They are also given a diary wherein they describe the taste of the yoghurt and any side-effects.

Before administration of the first dosage also waist-size, body weight and blood pressure are determined.

Blood samples are taken before consumption of the first daily dosage (1^{st} day). Glucose levels, glycated haemoglobin (HbA_{1c}) levels, lipid levels and metformin levels in the samples are determined.

After the 7^{th} day subjects are asked about any side-effects, such as gastro-intestinal problems.

On the 21^{st} day, blood glucose levels are determined again, in blood samples taken on an empty stomach. The subjects return the 21 (empty) yoghurt containers and sachets (to check for compliance) and are given 21 new containers containing the yoghurt and 21 sachets containing metformin or placebo.

On the 42^{nd} day, blood glucose, glycated haemoglobin (HbA_{1c}), lipid and metformin levels are determined in blood samples taken on an empty stomach. Further, waist-size, body weight and blood pressure are determined again. The subjects also return the containers and sachets.

Results after 42 Days (13 Subjects)

For practical reasons, the test has been started with a limited number of subjects (4 placebo, 10 treated with product according to the invention of which 6 in Trial group I, 4 in Trial group II). Provisional results for this initial group of subjects are:

Subject-compliance is in the subjects of trial groups I and II is excellent (100%), versus 75% in the placebo group. One out of four subjects in the placebo group stopped early, accordingly in the results herein below the number of subjects in the placebo group is 3).

The product taste of the product according to the invention is marked as at least as good as for the placebo as shown in Table 2, showing that the unfavourable metallic/bitter taste of metformin alone is masked in the yoghurt.

TABLE 2
Taste data after 42 days

| | Placebo | Trial group I | Trial group II |
|---|---|---|---|
| | (n = 3) | (n = 6) | (n = 4) |
| good | 2 | 3 | 3 |
| moderate | 0 | 2 | 1 |
| poor | 1 | 1 | 0 |
| bad | 0 | 0 | 0 |

With respect to side-effects the subjects of Trial groups I and II scored better than the subjects in the placebo group (see Table 3), This is surprising, since metformin alone is known to have disadvantageous side-effects, such as stomach complaints or diarrhoea.

TABLE 3
side-effects after 42 days

| | Placebo | Trial group I | Trial group II |
|---|---|---|---|
| | (n = 3) | (n = 6) | (n = 4) |
| reflux | 1 | 0 | 0 |
| stomach pain | 1 | 1 | 0 |
| abdominal pain | 0 | 0 | 0 |
| diarrhoea | 0 | 0 | 0 |
| fatigue | 1 | 0 | 0 |
| vertigo | 1 | 0 | 0 |

Metformin levels determined after 42 days (24 hours after last dose) were below the detection limit (0.25 mg/l) in all placebo subjects and most trial subjects. In the blood of one subject of Trial group I, metformin was still detectable (0.3 mg/l).

Table 4 shows the weight loss after 42 days. Compared to the placebo, the subjects with the product of the invention showed a 3-fold higher weight loss and a 2-3 fold higher reduction in waist size (indicating a reduction in visceral fat).

TABLE 4
weight loss after 42 days

| | Placebo | Trial group I | Trial group II |
|---|---|---|---|
| | (n = 3) | (n = 6) | (n = 4) |
| Δ weight (kg) | −0.6 | −1.9 | −1.9 |
| Δ waist size (cm) | −0.9 | −2.0 | −3.1 |

Table 5 illustrates the effect of the products of the invention on the prediabetic status.

TABLE 5
effect on prediabetic status after 42 days

| | Placebo | Trial group I | Trial group II |
|---|---|---|---|
| | (n = 3) | (n = 6) | (n = 4) |
| Hyperinsulinaemia | −5.5* | −1.4 | −3.9 |
| Internationale | | | |
| Unit | | | |
| insulin resistance | −0.9 | −0.13 | −3.9 |
| improvement | | | |
| (HOMA-2 | | | |
| fasting glucose | no change | −0.12 | −0.68 |
| (mmol/l) | | | |
| HbA1c (mmol/ml) | no change | −0.6 | −2.2 |

*high decrease is caused by results for one placebo subject, who had a detected decrease of 15; however, in that subject the glucose level increased, as an indication that this result may be a mistake in a measurement. In the other two placebo subjects no change was detected.

Further, it was found that in trial group II (450 mg metformin per day) the blood HDL concentration was increased (+0.12). No change was observed in the other two groups. A decrease in pulse was observed in all groups: −0.75 in the placebo group, −6 in trial group I and −2 in trial group II. And increase in HDL and/or decrease in pulse are desired in particular for prevention or therapeutic treatment of a cardiovascular disease.

Further, 4 out of 10 subjects in the trial group indicated to have a reduced appetite (1 in the placebo group).

### EXAMPLE 4

### Stability Test

Yoghurt is prepared as described in Example 3. A batch comprising no metformin (placebo), a batch comprising 200 mg metformin per 100 gram (low dosage) and a batch comprising 360 mg metformin (high dosage) were made.

Part of the batches were stored in a fridge (about 4° C.), other parts at room temperature (about 20° C.). Product stability was determined by measuring pH and the metformin content (measured amount as a percentage of the added amount), weekly for a period of 35 days. It was found that pH remained stable over this period (pH 4.2) at refrigerator temperature. Stability at room temperature was determined for only 35 days. Tables 6 and 7 show the measured amount of metformin in both products comprising metformin (relative to calculated amount=100%). No metformin was measured in the placebo.

TABLE 6
stored at about 4° C.:

| batch | day 0 | day 7 | day 14 | day 21 | day 28 | day 35 |
|---|---|---|---|---|---|---|
| low | 91 | 92 | 92 | 93 | 93 | 93 |
| dosage | | | | | | |
| high | 87 | 88 | 89 | 86 | 86 | 87 |
| dosage | | | | | | |

TABLE 7
stored at about 20° C.:

| batch | day 0 | day 7 | day 14 | day 21 | day 28 | day 35 |
|---|---|---|---|---|---|---|
| low | 91 | 95 | 94 | 93 | 92 | n.d. |
| dosage | | | | | | |
| high | 87 | 87 | 89 | 88 | 88 | n.d. |
| dosage | | | | | | |

These results illustrate that no substantial degradation of metformin takes place in a fermented product (which is acidic) over a period of 28 days (room temperature) or 35 days (refrigerator temperature) and that the metformin has no substantial effect on pH.

### EXAMPLE 5

### Bacteriological Test

A yoghurt drink (4% protein, 0% fat and 3.7% carbohydrate with an energy value of 31 kcal/100 g) was produced in manner as described above To this drink the desired amounts of metformin were added to 5 kg batches.

The yoghurt drink was divided into 125 g portions. The metformin level in the drinks was 0, 250 and 450 mg per serving of 125 g. The products were stored at about 4° C. for a shelf-life test. After 7 days storage the microbiological quality of the yoghurt drink portions was evaluated. No growth of micro-organisms was observed in the products and therefore they meet the required microbial specification

1. A nutritional product comprising a biguanide in a concentration of 20 to 1600 mg per 100 g, based on total weight of the nutritional product, the nutritional product further comprising at least one dietary component selected from the group of (i) digestible carbohydrates, (ii) amino acid sources, such as proteins, peptides, free amino acids including salts of free amino acids, (iii) lipids, and (iv) dietary fibre.

2. The nutritional product according to claim 1, wherein the biguanide is metformin.

3. The nutritional product according to claim 1, wherein the lipid content is 0-6 wt. %.

4. The nutritional product according to claim 1, wherein the product is packaged in a unit-dose packaging.

5. The nutritional product according to claim 4, wherein the nutritional product in the unit-dose package contains 50-500 mg biguanide.

6. (canceled)

7. The nutritional product according to claim 1, wherein the nutritional product is selected from the group of fermented milk products, unfermented milk products, fermented milk-substitute products, unfermented milk-substitute products, fruit drinks and baked cereal products.

8. The nutritional product according to claim 7, wherein the product is selected from the group of yoghurts, yoghurt-substitutes, including yoghurt drinks and yoghurt-substitute drinks.

9. The nutritional product according to claim 1, comprising vitamin D and/or a pectin.

10. (canceled)

11. Kit of parts, comprising a first unit dose packaging containing a nutritional product and a second unit dose packaging containing a biguanide, and optionally instructions for adding the biguanide to the nutritional product.

12. Kit of parts according to claim 11, wherein the second unit dosage packaging comprises 50-500 mg biguanide or wherein the second unit dosage packaging comprises 1000-3000 mg biguanide.

13. Kit of parts according to claim 11, wherein the nutritional product is selected from the group of fluid dairy products and fluid dairy-substitute products.

14. A method of prophylactically or therapeutically treating a subject with an aging-associated disorder comprising administering to the subject a nutritional product comprising a biguanide in a concentration of 20 to 1600 mg per 100 g, based on total weight of the nutritional product, the nutritional product further comprising at least one dietary component selected from the group of (i) digestible carbohydrates, (ii) amino acid sources, such as proteins, peptides, free amino acids including salts of free amino acids, (iii) lipids, and (iv) dietary fibre.

15. The method according to claim 14, wherein the aging associated disorder is insulin resistance, diabetes type II, metabolic syndrome, obesity, hypertension, hypercholesterolaemia, dyslipaedea, atherosclerosis, steotosis, lung carcinoma, liver carcinoma, colonic carcinoma, polycystic ovary syndrome, or hyperinsulaemia.

16. The method according to claim 14, wherein the aging associated disorder is insulin resistance or hyperinsulaemia.

17. The method according to claim 14, wherein the treating results in reducing visceral fat content or waist size of a subject.

18. The method according to claim 14, wherein the subject is a non-diabetic subject.

19. (canceled)

20. The method according to claim 14 wherein the subject is a human subject having a body mass index of 26.0 kg/m2 or more.

21. (canceled)

22. (canceled)

23. The method according to claim 14, wherein the daily dosage of the biguanide to be administered is in the range of 50-500 mg.

24. The method according to claim 14, wherein the biguanide is metformin.

25. A method for preparing a nutritional product according to claim 8, wherein yoghurt or substitute-yoghurt is mixed with a biguanide stabiliser, thereafter adding the biguanide thereby obtaining the yoghurt or substitute-yoghurt comprising biguanide, and thereafter aseptically packaging the yoghurt or substitute-yoghurt comprising biguanide and biguanide stabiliser.
